(19) United States
(12) Patent Application Publication
(10) Pub. No.: US 2008/0318926 A1
(43) Pub. Date: Dec. 25, 2008

(54) Methods of Treating Mood Disorders Using Pyridyloxymethyl and Benzisoxazole Azabicyclic Derivatives

(75) Inventors: Kathleen S. Ice, New London, CT (US); Peter J. Snyder, Marlborough, CT (US); Eric J. Watsky, Niantic, CT (US)

Correspondence Address: PFIZER INC;Steve T. Zelson, 150 EAST 42ND STREET, 5TH FLOOR - STOP 49, NEW YORK, NY, 10017-5612, US

(73) Assignee: Pfizer Inc

(21) Appl. No.: 11/574,938

(22) PCT Filed: Sep. 1, 2005
(86) PCT No.: PCT/IB05/02913

§ 371 Date: Mar. 8, 2007

Related U.S. Application Data
(60) Provisional application No. 60/608,561, filed on Sep. 10, 2004.

(51) Int. Cl. A61K 31/397; A61K 31/4985; A61P 25/00; A61K 31/5377

(52) U.S. Cl. 514/210.21; 514/249; 514/233.2

(57) ABSTRACT

An aminomethylpyridyloxymethyl/benzisoxazole substituted azabicyclic compound, a pharmaceutical composition comprising same, and a method of treating a mood disorder selected from the group consisting of Somatization Disorder, Borderline Personality Disorder, Narcissistic Personality Disorder, Suicidal Ideation, and Antisocial Personality Disorder.

### FIELD OF THE INVENTION

The invention pertains to methods of treating certain mood disorders by administering aminomethylpyridyloxymethyl/benzisoxazole substituted azabicyclic compounds that, among other things, can singly serve as an effective 5-HT1B, 5-HT2A, and D2 receptor inhibitor, e.g. antagonist, inverse agonist, and/or partial agonist. The invention also relates to methods of treating mood disorders comprising administering a said compound with a mood drug.

### BACKGROUND OF THE INVENTION

Mood disorders can be medically treated in various ways. Of increasing importance in this regard are psychotropic drugs. But while such drugs have therapeutic effect, they also may cause unwanted and serious side effects. For example, mood disorders can be treated with so-called typical drugs, which have been theorized to block certain dopamine (D2) receptors in the brain thought responsible for the positive symptoms of delusions, disordered thinking and the like. However, while these drugs can ameliorate some of the positive symptoms, they can also adversely affect the motor system, causing muscle problems such as spasms, cramps, tremors and Parkinsonism. Inasmuch as these types of side effects—generally characterized as Extrapyramidal Symptoms (EPS)—can be severe enough to disrupt daily activities, resort has been made to so-called atypical drugs.

Atypical antipsychotics have reduced incidents of EPS and can alleviate symptoms of mood disorders. Although antipsychotic drugs are believed to be more selective in their chemical effect on the brain, thereby reducing EPS, they too may have side effects. Such side effects can include Male or Female Sexual Dysfunction. While these are not often as disruptive as those presented by typical drug therapy, they may nonetheless be of consequence to the patient. For example, atypical drugs can be sedating and can cause weight gain.

The situation is further complicated when several mood disorders are present in a patient. In such cases, treatment often entails the administration of a combination of drugs, e.g., two different mood disorders. Because each such drug has its own side effects, the combined administration can lead to a multiplication or enhancement of same, all to the detriment of the patient. Moreover, it is theorized that different brain receptors, such as D2, 5HT-2A, and 5HT-1B, or combination or permutation of said receptors, are somehow implicated in Mood Disorders. A class of aminomethylphenoxymethyl/benzisoxazole substituted azabicyclic compounds, useful as selective agonists and antagonists of serotonin 1 (5-HT1) receptors, is described in WO 99/52907 to Bright, which is incorporated herein by reference.

It has hitherto proven difficult to find a single drug that can treat a patient suffering from Mood Disorders where a plurality of different receptors are in play. Accordingly, there is an on-going need for a psychotropic drug that has a pronounced reduction in side effects, such as Female Sexual Dysfunction, and that can efficaciously and by itself treat these disorders in which an antagonist or agonist to different receptors is indicated. Specifically, it would be desirable to find a drug that can treat Mood Disorders in which D2, 5HT-2A, and 5HT-1B receptors are involved.

The present invention addresses the aforementioned needs.

### SUMMARY OF THE INVENTION

In one practice, the invention relates to a method of treating a mood disorder selected from the group consisting of Somatization Disorder, Borderline Personality Disorder, Narcissistic Personality Disorder, Suicidal Ideation, and Antisocial Personality Disorder in a mammal comprising administering to said mammal a compound having Formula I:

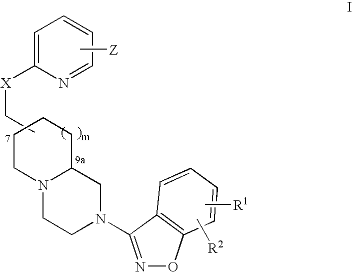

and pharmaceutically acceptable salts or solvates thereof, wherein

m is 0 or 1;

Z is

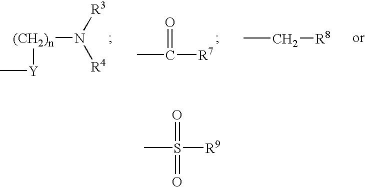

R⁷ is hydrogen or (C₁-C₃)alkoxy;

R⁸ is hydrogen, hydroxy, or (C₁-C₃)alkoxy;

R⁹ is (C₁-C₃)alkoxy;

X is oxygen or NR, wherein R is hydrogen or (C₁-C₆)alkyl;

Y is methylene when n is 0, 1 or 2;

or Y is oxygen, nitrogen or sulfur, when n is 2, 3 or 4;

R¹ and R² are each independently hydrogen, halogen, or a (C₁-C₆)alkyl, (C₁-C₆)alkoxy or a (C₁-C₆)alkoxy(C₁-C₆)alkyl group, wherein any one of which (C₁-C₆)alkyl, (C₁-C₆)alkoxy or (C₁-C₆)alkoxy(C₁-C₆)alkyl groups may be unsubstituted or substituted with one or more halogens;

R³ and R⁴ are each independently hydrogen, a (C₁-C₆)alkyl, a (C₃-C₇)cycloalkyl, or a 5 to 6 membered heterocyclic group, wherein any one of which (C₁-C₆)alkyl, (C₃-C₇)cycloalkyl, or 5 to 6 membered heterocyclic groups may be unsubstituted or substitituted with one or more substituents selected from the group consisting of (C₁-C₄)alkyl, (C₃-C₇)cycloalkyl, (C₁-C₄)alkoxy, (C₆-C₁₀)aryl, a 5 to 6 member heterocyclic, amino, halogen and hydroxy groups; or

R³ and R⁴, together with the nitrogen atom to which they are attached, form:

(i) a 3 to 7 membered saturated or unsaturated monocyclic ring; or

(ii) a 4 to 10 membered saturated or unsaturated polycyclic ring,

wherein said monocyclic or polycyclic ring optionally has one or two heteroatoms selected from nitrogen, oxygen and sulfur; and

wherein any of said rings (i) or (ii) may be unsubstituted or substituted with one or more (C₁-C₄)alkyl, (C₁-C₄)alkoxy, (C₁-C₄)alkoxy(C₁-C₄)alkyl, (C₃-C₇)cycloalkyl, (C₆-C₁₀)aryl, (C₇ to C₁₃)aralkyl, a 5 to 10 membered heteroaryl, hydroxy, amino, cyano, or halogen groups.

In a particular embodiment, the compound of the invention has the formula:

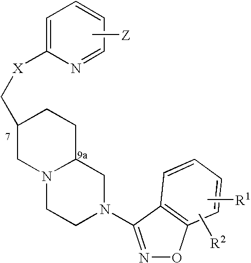

or a pharmaceutically acceptable salt or solvate thereof, wherein Z is independently any one or combination of the following:

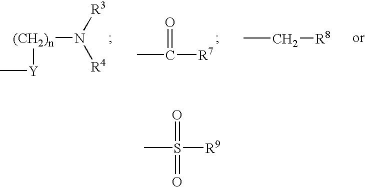

wherein R⁷ is hydrogen or (C₁-C₃)alkoxy;

R⁸ is hydrogen, hydroxy, or (C₁-C₃)alkoxy;

R⁹ is (C₁-C₃)alkoxy;

X is oxygen or NR, wherein R is hydrogen or (C₁-C₆)alkyl;

Y is methylene when n is 0, 1 or 2; or oxygen, nitrogen or sulfur when n is 2, 3 or 4;

R¹ and R² are each independently hydrogen, halogen, or a (C₁-C₆)alkyl, (C₁-C₆)alkoxy or a (C₁-C₆)alkoxy(C₁-C₆)alkyl group, wherein any one of which (C₁-C₆)alkyl, (C₁-C₆)alkoxy or (C₁-C₆)alkoxy(C₁-C₆)alkyl groups may be unsubstituted or substituted with one or more halogens;

R³ and R⁴ are each independently hydrogen, a (C₁-C₆)alkyl, a (C₃-C₇)cycloalkyl, or a 5 to 6 membered heterocyclic group, wherein any one of which (C₁-C₆)alkyl, (C₃-C₇)cycloalkyl, or 5 to 6 membered heterocyclic groups may be unsubstituted or substitituted with one or more of any of the following: (C₁-C₄)alkyl, (C₃-C₇)cycloalkyl, (C₁-C₄)alkoxy, (C₆-C₁₀)aryl, a 5 to 6 member heterocyclic, amino, halogen or hydroxy groups; or

R³ and R⁴ together with the nitrogen atom to which they are attached form:

(i) a 3 to 7 membered saturated or unsaturated monocyclic ring; or

(ii) a 4 to 10 membered saturated or unsaturated polycyclic ring,

wherein said monocyclic or polycyclic ring optionally has one or two additional heteroatoms selected from nitrogen, oxygen and sulfur,

wherein any of said rings (i) or (ii) may be unsubstituted or substituted with one or more substituents selected from (C₁-C₄)alkyl, (C₁-C₄)alkoxy, (C₁-C₄)alkoxy(C₁-C₄)alkyl, (C₃-c₇)cycloalkyl, (C₆-C₁₀)aryl, (C₇ to C₁₃)aralkyl, a 5 to 10 membered heteroaryl, hydroxy, amino, cyano, and halogen groups.

In another aspect, the invention relates to a method of treating mood disorders selected from the group consisting of Somatization Disorder, Borderline Personality Disorder, Narcissistic Personality Disorder, Suicidal Ideation, and Antisocial Personality Disorder, in a mammal, wherein said mammal is in need of said treatment, comprising administering to said mammal a pharmaceutical composition comprising a compound of Formula I, wherein a ligand, for example, an antagonist, partial agonist (with 80% or more antagonism), or inverse agonist, to D2, 5HT-2A, and 5HT-1B receptors, individually or any combination thereof (including at least two receptors and also three receptors), is indicated. In another aspect, the compound of Formula I manifests a ratio of D2:5HT1B receptor binding of about 20 or less; and/or inhibitory activity to each of said D2, 5HT1B, and 5HT2A receptors.

In another aspect, the invention relates to a method of treating any of the aforementioned mood disorders in a mammal comprising administering to said mammal a pharmaceutical composition comprising a compound of Formula I in combination with another mood drug. Non-limiting examples of mood drugs include Norepinephrine Reuptake Inhibitors (NRI), corticotropin-releasing hormone (CRH) antagonists, and Selective Serotonin Reuptake Inhibitors (SSRI).

The compounds of Formula I have receptor binding activity to at least two and preferably all three of the D2, 5HT1B, and 5HT2A receptors. The level of inhibition in this regard is such that the compound of the invention is therapeutically effective to treat a mood disorder selected from the group consisting of Somatization Disorder, Borderline Personality Disorder, Narcissistic Personality Disorder, Suicidal Ideation, and Antisocial Personality Disorder in a mammal wherein activity against all of these receptors is indicated. This means that the compound of Formula I has an effective Ki of less than or equal to 20 nM at at least two and preferably all three receptors. This would apply to any Ki less than 20 nM, for example, 15 nM.

Furthermore, the compounds of Formula I have an intrinsic efficacy of an antagonist and/or inverse agonist at human D2, human 5HT1B, and human 5HT2A receptors. The intrinsic efficacy as measured by adenylate cyclase activity, phosphoinositol turnover, or other methods known in the art. The compounds of Formula I have an intrinsic efficacy of an antagonist and/or inverse agonist at human D2 and human 5HT2A receptors and an intrinsic efficacy of an antagonist, partial agonist (with 80% or more antagonism) at human 5HT1B receptors. As above, the intrinsic efficacy can be measured by adenylate cyclase activity or phosphoinositol turnover.

The compounds of Formula I preferably have a functional Ki value at 5HT1B of less than or equal to 5 nM in combination with a functional Ki value of less than or equal to 20 nM at human D2 and human 5HT2A receptors.

In another aspect, the invention relates to a method of treating a mood disorder selected from the group consisting of Somatization Disorder, Borderline Personality Disorder, Narcissistic Personality Disorder, Suicidal Ideation, and Antisocial Personality Disorder in a mammal comprising administering to said mammal a therapeutically effective amount of a compound which has at least 80% antagonism, or inverse agonist to each of D2, 5HT1B and 5HT2A receptors, wherein said mammal is in need of said treatment.

In another aspect, the invention relates to a method of treating a mood disorder selected from the group consisting of Somatization Disorder, Borderline Personality Disorder, Narcissistic Personality Disorder, Suicidal Ideation, and Antisocial Personality Disorder in a mammal comprising administering to said mammal a therapeutically effective amount of a D2, 5HT1B inhibitor having effective inhibitory activity with an in vivo effective Ki of no more than 15 nM at each of said receptors, wherein said mammal is in need of such said treatment.

Furthermore, compounds of Formula I preferably can singly show in vivo efficacy in animal models of 5HT1B, D2, and 5HT2A antagonism or inverse agonism. Representative animal models include the following examples but are not limited to such models. Another aspect of the present invention is where the compounds have the preferred ID₅₀ of at least two of the models. Compounds are tested for their ability to antagonize the hypothermia response produced by a 5-HT1B agonist as a measure of in vivo 5HT1B antagonist activity. Compounds or vehicle are administered to guinea pigs, 0 to 60 minutes subcutaneous (sc) prior to the 5HT1B agonist and body temperatures are monitored over a four hour period after agonist administration. The present invention preferably comprises compounds with an ID₅₀ of less than or equal to 1 mg/kg, sc in hypothermia.

In another animal model, compounds are tested for their ability to antagonize DOI (drug interaction)-induced head twitches as a measure of in vivo 5HT2A antagonist activity. Administration of the 5HT2A agonist, DOI, elicits a characteristic head shaking behavior (head twitch) that has been attributed to activation of 5HT2A receptors. Compounds or vehicle are administered to habituated rats, 30 to 60 minutes sc prior to 3.2 mg/kg DOI, and head twitches are counted over a 30-minute test period. The invention preferably comprises compounds with an ID₅₀ of less than or equal to 10 mg/kg, sc in 5HT2A head twitch.

In addition, compounds are tested for their ability to antagonize d-amphetamine-induced hyperactivity as a measure of in vivo dopamine D2 receptor antagonist activity. Administration of low doses of the indirect dopamine agonist, d-amphetamine, produces a dramatic increase in horizontal locomotor activity in rats, a phenomenon which has been attributed to activation of the mesolimbic dopamine system, and which therefore provides a rodent model of the hyperdopaminergic activity implicated in schizophrenia. Compounds or vehicle are administered to habituated rats, 30-60 minutes s.c. prior to 1.0 mg/kg of d-amphetamine SO₄, and locomotor activity data are recorded in computer-monitored activity chambers for the 3-hour duration of the hyperactivity response. The compound of Formula I includes compounds with an ID₅₀ of less than or equal to 10 mg/kg, sc in d-amphetamine locomotor activity.

### DETAILED DESCRIPTION OF THE INVENTION

In one embodiment, the invention relates to a method of treating a mood disorder selected from the group consisting of Somatization Disorder, Borderline Personality Disorder, Narcissistic Personality Disorder, Suicidal Ideation, and Antisocial Personality Disorder in a mammal comprising administering to said mammal a compound of Formula I described hereinabove. The compound of Formula I has inter alia binding activity to one or multiple receptors, including D2, 5HT1B, and 5HT2A receptors, individually or in any combination thereof. In a preferred embodiment, the compound of Formula I has binding activity (based on e.g. IC₅₀ or Ki) to D2 and 5HT1B receptors in a ratio of D2:5HT1B of about 20 or less; in more preferred practices, this ratio is about 10 or less; about 5 or less; most preferably about 1.

The compound of Formula I is a psychotropic drug that can treat, in addition to the mood disorders described herein, psychosis and other CNS disorders as described in U.S. Ser. No. 10/800,328, now Published Patent Application 2005/26922A1. The entire contents of U.S. Ser. No. 10/800,328 are incorporated herein by reference.

Unless otherwise indicated, the term “inhibitory activity” and related variations of the same as used herein means that the compound serves, without limitation, as an antagonist, inverse agonist and/or partial agonist (80% antagonism or more) and the like to any of the receptors indicated herein; for example, the compound exhibits a binding affinity with a Ki of about 1 micromolar or less, with preferred practices having a Ki of about 100 nanomolar (nM) or less, about 50 nM or less, about 20 nM or less, about 15 nM or less, and most preferably about 10 nM or less, for any of the receptors aforesaid.

In an exemplifying embodiment, the compound of Formula I includes pharmaceutically acceptable salts thereof, (e.g. acid addition salts and base addition salts) and prodrugs and solvates thereof. Without limitation, examples of pharmaceutically acceptable acid addition salts of the compounds of Formula I are the salts of hydrochloric acid, p-toluenesulfonic acid, fumaric acid, citric acid, succinic acid, salicylic acid, oxalic acid, hydrobromic acid, phosphoric acid, methanesulfonic acid, tartaric acid, malate, di-p-toluoyl tartaric acid, and mandelic acid. Other possible acid addition salts are, e.g., salts containing pharmaceutically acceptable anions, such as the hydroiodide, nitrate, sulfate or bisulfate, phosphate or acid phosphate, acetate, lactate, gluconate, saccharate, benzoate, methanesulfonate, ethanesulfonate, benzenesulfonate, and pamoate (i.e., 1.1′-methylene-bis-(2-hydroxy-3-naphthoate) salts).

The compound of Formula I may have optical centers (e.g., at the 7 and 9a positions indicated) and thus may occur in different enantiomeric configurations. The compounds of Formula I includes all enantiomers, diastereomers, and other stereoisomers and optical isomers of such compound of Formula I, as well as racemic and other mixtures thereof. For example, the compounds of Formula I includes (R) and (S) enantiomers and cis and trans isomers. The present invention further includes all radiolabelled forms of the compounds of Formula I. Preferred radiolabelled compounds are those wherein the radiolabels are selected from ³H, ¹¹C, ¹⁴C, ¹⁸F, ¹²³I and ¹²⁵I. Such radiolabelled compounds are useful as research and diagnostic tools in metabolism pharmacokinetics studies and in binding assays in animals and man. In another embodiment, the invention is directed to a compound of Formula I wherein in an assay of D2, 5HT1B, or 5HT2A binding, said compound exhibits a Ki with intrinsic efficacy of about 1 micromolar or less; preferably exhibiting Ki's of about 100 nanomolar (nM) or less, about 50 nM or less, about 20 nM or less, about 15 nM or less, and most preferably about 10 nM or less. The assays in this regard are those known in or adaptable from the art.

The present invention includes a method of treating a mood disorder selected from the group consisting of Somatization Disorder, Borderline Personality Disorder, Narcissistic Personality Disorder, Suicidal Ideation, and Antisocial Personality Disorder in a mammal comprising administering to said mammal a therapeutically effective amount of a compound of formula I as described herein. The present invention further includes manufacture a medicament containing a therapeutically effective amount of compound of formula I for the treatment of a mood disorder selected from the group consisting of Somatization Disorder, Borderline Personality Disorder, Narcissistic Personality Disorder, Suicidal Ideation, and Antisocial Personality Disorder in a mammal.

In a preferred embodiment, the compound of Formula I has the following structure:

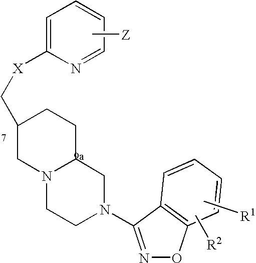

wherein Z is

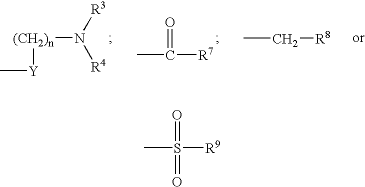

X is oxygen; n is 0; R¹ is hydrogen; R² is hydrogen or halogen; and R³ is hydrogen or a (C₁-C₃)alkyl.

In another preferred embodiment, R² is hydrogen; R³ is hydrogen; and R⁴ is

a) a (C₁-C₆)alkyl group;

b) a (C₃-C₇)cycloalkyl group; or

c) a 5 to 6 member heterocyclic group, wherein any one of which groups a), b) or c) may be unsubstituted or substitituted with one or more of any of the following: (C₁-C₄)alkyl, (C₃-C₇)cycloalkyl, (C₁-C₄)alkoxy, (C₆-C₁₀)aryl, a 5 to 6 member heterocyclic, amino, halogen or hydroxy groups.

In another preferred embodiment, Z is

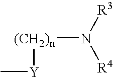

Y is methylene; and R⁴ is

a) a (C₁-C₄)alkyl which may be unsubstituted or substituted with one of the following: phenyl, cyclopropyl, methoxy, or substituted with a 5 to 6 membered heterocyclic, said heterocyclic having at least one nitrogen or oxygen atom;

b) an unsubstituted (C₃-C₇)cycloalkyl; or

c) a 5 to 6 membered heterocyclic which can be unsubstituted or substituted with a (C₁-C₃)alkyl or a (C₁-C₃)alkoxy, said 5 to 6 member heterocyclic c) having at least one nitrogen atom and up to one other heteroatom selected from nitrogen, oxygen and sulfur.

In another preferred embodiment, R⁴ is

a) an unsubstituted C₄ alkyl; a C₃ alkyl substituted with methoxy; a (C₁-C₂)alkyl substituted with phenyl or cyclopropyl; a (C₁-C₂)alkyl substituted with a 5 membered heterocyclic having a nitrogen or oxygen atom; or a (C₁-C₂)alkyl substituted with a 6 membered heterocyclic having at least one nitrogen;

b) an unsubstituted cyclopropyl; or

c) a 5 to 6 membered ring which can be unsubstituted or substituted with a methyl or methoxy, said 5 to 6 membered ring c) having at least one nitrogen atom and up to one other heteroatom selected from nitrogen, oxygen and sulfur, said (C₁-C₃)alkyl is methyl and said (C₁-C₃)alkoxy is methoxy.

In another preferred embodiment, R² is hydrogen; R³ is (C₁-C₃)alkyl; and R⁴ is

a) a (C₁-C₄)alkyl group; or

b) a (C₅-C₆)cycloalkyl group, wherein either of which groups a) or b) may be unsubstituted or substituted with one or more (C₁-C₃)alkoxy or amino groups.

In another preferred embodiment, the amino has the formula —NR⁵R⁶ wherein R⁵ and R⁶ are each independently hydrogen or (C₁-C₃)alkyl.

In another preferred embodiment, R⁴ is

a) a (C₁-C₄)alkyl group unsubstituted or substituted with one or more methoxy or amino groups wherein R⁵ is hydrogen and R⁶ is methyl; or

b) an unsubstituted (C₅-C₆)cycloalkyl group.

In another preferred embodiment, Z is

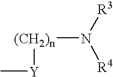

wherein Y is methylene; X is oxygen; n is 0; R¹ is hydrogen; R² is hydrogen; and R³ and R⁴ together with the nitrogen atom to which they are attached form i) a saturated non-aromatic 3 to 7 membered monocyclic ring, said ring i) being unsubstituted or substituted with one or more (C₁-C₄)alkyl, (C₁-C₄)alkoxy(C₁-C₄)alkyl, or hydroxy groups.

In another preferred embodiment, Z is

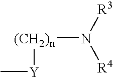

wherein Y is methylene; X is oxygen; n is 0; R¹ is hydrogen; R² is hydrogen; and R³ and R⁴ together with the nitrogen atom to which they are attached form an unsubstituted 5 to 6 membered heterocyclic ring, which heterocyclic ring, in addition to the nitrogen atom to which R³ and R⁴ are attached, has one additional nitrogen atom, or one sulfur atom, or one oxygen atom.

In another preferred embodiment, Z is

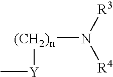

wherein Y is methylene; n is 0; R² is halogen; and R⁴ is

a) a (C₁-C₅)alkyl;

b) a (C₃-C₆) cycloalkyl group, wherein any of which groups a) or b) can be unsubstituted or substituted with one or more of any of the following: cyclopropyl; halogen; hydroxy; a 5 to 6 membered heterocyclic group wherein said 5 to 6 membered heterocyclic group may be unsubstituted or substituted with one or more methyl groups; or phenyl wherein said phenyl may be unsubstituted or substituted with one or more halogens; or R⁴ is

c) a 5 membered heterocyclic group.

In another preferred embodiment, R² is fluorine; and R³ is hydrogen or methyl.

In another preferred embodiment, R² is halogen; and R³ and R⁴ together with the nitrogen atom to which they are attached form

i) a saturated 3 to 7 membered monocyclic ring, which monocylcic ring may be unsubstituted or substituted with one or more phenyl, (C₁-C₃)alkyl, or (C₁₋₄)alkoxy(C₁₋₄)alkyl groups; or

ii) a 5 to 6 membered monocyclic ring, which ring may be unsubstituted or substituted with one or more (C₁-C₃) alkyl groups, and which ring has one additional nitrogen or one oxygen atom.

The following preferred compounds were representatively observed to exhibit a Ki value of about 20 nM or less for at least two of the following receptors: D2, 5HT1B and 5HT2A or an effective Ki value at about 10 nM or less for each of said receptors. The non-limiting examples of the compound Formula I include compounds independently selected from any one or more than one compound from the group consisting of:

(7R, 9aS)-trans-2-Benzo[d]isoxazol-3-yl-7-(6-morpholin-4-ylmethyl-pyridin-2-yloxymethyl)-octahydro-pyrido[1,2-a]pyrazine;

(7R, 9aS)-trans-2-Benzo[d]isoxazol-3-yl-7-(5-piperidin-1-ylmethyl-pyridin-2-yloxymethyl)-octahydro-pyrido[1,2-a]pyrazine;

(7R, 9aS)-trans-2-(5-Fluoro-benzo[d]isoxazol-3-yl)-7-(5-pyrrolidin-1-ylmethyl-pyridin-2-yloxymethyl)-octahydro-pyrido[1,2-a]pyrazine;

(7R, 9aS)-trans-[6-(2-Benzo[d]isoxazol-3-yl-octahydro-pyrido[1,2-a]pyrazin-7-ylmethoxy)-pyridin-3-ylmethyl]-diethyl-amine;

(7R, 9aS)-trans-[6-(2-Benzo[d]isoxazol-3-yl-octahydro-pyrido[1,2-a]pyrazin-7-ylmethoxy)-pyridin-3-ylmethyl]-dimethyl-amine;

(7R, 9aS)-trans-[6-(2-Benzo[d]isoxazol-3-yl-octahydro-pyrido[1,2-a]pyrazin-7-ylmethoxy)-pyridin-3-ylmethyl]-ethyl-methyl-amine;

(7R, 9aS)-trans-[6-(2-Benzo[d]isoxazol-3-yl-octahydro-pyrido[1,2-a]pyrazin-7-ylmethoxy)-pyridin-3-ylmethyl]-(2-methoxy-1-methyl-ethyl)-amine;

(7R, 9aS)-trans-[6-(2-Benzo[d]isoxazol-3-yl-octahydro-pyrido[1,2-a]pyrazin-7-ylmethoxy)-pyridin-3-ylmethyl]-(2-methoxy-ethyl)-methyl-amine;

(7R, 9aS)-trans-[6-(2-Benzo[d]isoxazol-3-yl-octahydro-pyrido[1,2-a]pyrazin-7-ylmethoxy)-pyridin-3-ylmethyl]-cyclopentyl-methyl-amine;

(7R, 9aS)-trans-7-(5-Azetidin-1-ylmethyl-pyridin-2-yloxymethyl)-2-benzo[d]isoxazol-3-yl-octahydro-pyrido[1,2-a]pyrazine;

(7R, 9aS)-trans-2-Benzo[d]isoxazol-3-yl-7-[5-(2-methyl-aziridin-1-ylmethyl)-pyridin-2-yloxymethyl]-octahydro-pyrido[1,2-a]pyrazine;

(7R, 9aS)-trans-2-Benzo[d]isoxazol-3-yl-7-[5-(2-methoxymethyl-pyrrolidin-1-ylmethyl)-pyridin-2-yloxymethyl]-octahydro-pyrido[1,2-a]pyrazine;

(7R, 9aS)-trans-[6-(2-Benzo[d]isoxazol-3-yl-octahydro-pyrido[1,2-a]pyrazin-7-ylmethoxy)-pyridin-3-ylmethyl]-tert-butyl-amine;

(7S, 9aS)-cis-[6-(2-Benzo[d]isoxazol-3-yl-octahydro-pyrido[1,2-a]pyrazin-7-ylmethoxy)-pyridin-3-ylmethyl]-ethyl-methyl-amine;

(7S, 9aS)-cis-7-(5-Azetidin-1-ylmethyl-pyridin-2-yloxymethyl)-2-benzo[d]isoxazol-3-yl-octahydro-pyrido[1,2-a]pyrazine;

(7R, 9aS)-trans-[6-(2-Benzo[d]isoxazol-3-yl-octahydro-pyrido[1,2-a]pyrazin-7-ylmethoxy)-pyridin-2-ylmethyl]-dimethyl-amine;

(7R, 9aS)-trans-Cyclohexyl-{6-[2-(5-fluoro-benzo[d]isoxazol-3-yl)-octahydro-pyrido[1,2-a]pyrazin-7-ylmethoxy]-pyridin-2-ylmethyl}-amine;

(7R, 9aS)-trans-2-(Ethyl-{6-[2-(5-fluoro-benzo[d]isoxazol-3-yl )-octahydro-pyrido[1,2-a]pyrazin-7-ylmethoxy]-pyridin-2-ylmethyl}-amino)-ethanol;

(7R, 9aS)-trans-7-[6-(2,6-Dimethyl-piperidin-1-ylmethyl)-pyridin-2-yloxymethyl]-2-(5-fluoro-benzo[d]isoxazol-3-yl )-octahydro-pyrido[1,2-a]pyrazine;

(7R, 9aS)-trans-(1,2-Dimethyl-propyl)-{6-[2-(5-fluoro-benzo[d]isoxazol-3-yl)-octahydro-pyrido[1,2-a]pyrazin-7-ylmethoxy]-pyridin-2-ylmethyl}-amine;

(7R, 9aS)-trans-[6-(2-Benzo[d]isoxazol-3-yl-octahydro-pyrido[1,2-a]pyrazin-7-ylmethoxy)-pyridin-2-ylmethyl]-(2-methoxy-ethyl)-methyl-amine;

(7R, 9aS)-trans-1-[6-(2-Benzo[d]isoxazol-3-yl-octahydro-pyrido[1,2-a]pyrazin-7-ylmethoxy)-pyridin-3-ylmethyl]-(S)-pyrrolidin-3-ol;

(7R, 9aS)-trans-1-[6-(2-Benzo[d]isoxazol-3-yl-octahydro-pyrido[1,2-a]pyrazin-7-ylmethoxy)-pyridin-3-ylmethyl]-(R)-pyrrolidin-3-ol;

(7R, 9aS)-trans-2-Benzo[d]isoxazol-3-yl-7-[5-(2-methyl-pyrrolidin-1-ylmethyl)-pyridin-2-yloxymethyl]-octahydro-pyrido[1,2-a]pyrazine;

(7R, 9aS)-trans-1-[6-(2-Benzo[d]isoxazol-3-yl-octahydro-pyrido[1,2-a]pyrazin-7-ylmethoxy)-pyridin-3-ylmethyl]-piperidin-4-ol;

(7R, 9aS)-trans-Cyclopropyl-{6-[2-(5-fluoro-benzo[d]isoxazol-3-yl)-octahydro-pyrido[1,2-a]pyrazin-7-ylmethoxy]-pyridin-2-ylmethyl}-amine;

(7R, 9aS)-trans-Cyclopropylmethyl-{6-[2-(5-fluoro-benzo[d]isoxazol-3-yl)-octahydro-pyrido[1,2-a]pyrazin-7-ylmethoxy]-pyridin-2-ylmethyl}-amine;

(7R, 9aS)-trans-2-(5-Fluoro-benzo[d]isoxazol-3-yl)-7-[6-(4-methyl-piperazin-1-ylmethyl)-pyridin-2-yloxymethyl]-octahydro-pyrido[1,2-a]pyrazine;

(7R, 9aS)-trans-2-(5-Fluoro-benzo[d]isoxazol-3-yl)-7-(6-piperidin-1-ylmethyl-pyridin-2-yloxymethyl)-octahydro-pyrido[1,2-a]pyrazine;

(7R, 9aS)-trans-{6-[2-(5-Fluoro-benzo[d]isoxazol-3-yl)-octahydro-pyrido[1,2-a]pyrazin-7-ylmethoxy]-pyridin-2-ylmethyl}-dimethyl-amine;

(7R, 9aS)-trans-{6-[2-(5-Fluoro-benzo[d]isoxazol-3-yl)-octahydro-pyrido[1,2-a]pyrazin-7-ylmethoxy]-pyridin-2-ylmethyl}-(tetrahydro-furan-2-ylmethyl)-amine;

(7R, 9aS)-trans-7-[6-(2,5-Dimethyl-pyrrolidin-1-ylmethyl)-pyridin-2-yloxymethyl]-2-(5-fluoro-benzo[d]isoxazol-3-yl)-octahydro-pyrido[1,2-a]pyrazine;

(7R, 9aS)-trans-{6-[2-(5-Fluoro-benzo[d]isoxazol-3-yl)-octahydro-pyrido[1,2-a]pyrazin-7-ylmethoxy]-pyridin-2-ylmethyl}-[3-(4-methyl-piperazin-1-yl)-propyl]-amine;

(7R, 9aS)-trans-{6-[2-(5-Fluoro-benzo[d]isoxazol-3-yl)-octahydro-pyrido[1,2-a]pyrazin-7-ylmethoxy]-pyridin-2-ylmethyl}-pyrrolidin-1-yl-amine;

(7R, 9aS)-trans-7-(6-Azepan-1-ylmethyl-pyridin-2-yloxymethyl )-2-(5-fluoro-benzo[d]isoxazol-3-yl)-octahydro-pyrido[1,2-a]pyrazine;

(7S, 9aS)-cis-[6-(2-Benzo[d]isoxazol-3-yl-octahydro-pyrido[1,2-a]pyrazin-7-ylmethoxy)-pyridin-3-ylmethyl]-cyclohexyl-methyl-amine;

(7R, 9aS)-trans-1-[6-(2-Benzo[d]isoxazol-3-yl-octahydro-pyrido[1,2-a]pyrazin-7-ylmethoxy)-pyridin-2-ylmethyl]-(S)-pyrrolidin-3-ol;

(7R, 9aS)-trans-1-[6-(2-Benzo[d]isoxazol-3-yl-octahydro-pyrido[1,2-a]pyrazin-7-ylmethoxy)-pyridin-2-ylmethyl]-(R)-pyrrolidin-3-ol;

(7R, 9aS)-trans-2-Benzo[d]isoxazol-3-yl-7-(6-pyrrolidin-1-ylmethyl-pyridin-2-yloxymethyl)-octahydro-pyrido[1,2-a]pyrazine;

(7R, 9aS)-trans-[6-(2-Benzo[d]isoxazol-3-yl-octahydro-pyrido[1,2-a]pyrazin-7-ylmethoxy)-pyridin-2-ylmethyl]-benzyl-amine;

(7R, 9aS)-trans-2-Benzo[d]isoxazol-3-yl-7-(5-pyrrolidin-1-ylmethyl-pyridin-2-yloxymethyl)-octahydro-pyrido[1,2-a]pyrazine; and

(7S, 9aS)-cis-2-Benzo[d]isoxazol-3-yl-7-(5-pyrrolidin-1-ylmethyl-pyridin-2-yloxymethyl)-octahydro-pyrido[1,2-a]pyrazine.

The present invention includes a method of treating a mood disorder selected from the group consisting of Somatization Disorder, Borderline Personality Disorder, Narcissistic Personality Disorder, Suicidal Ideation, and Antisocial Personality Disorder in a mammal comprising administering to said mammal a therapeutically effective amount of compound of Formula I having the following structure:

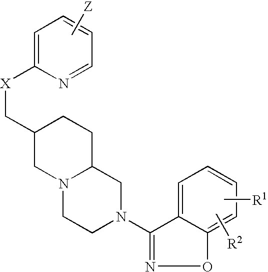

or a pharmaceutically acceptable salt or solvate thereof,

wherein X is oxygen or NR, wherein R is hydrogen or (C₁-C₆)alkyl;

R¹ and R² are each independently hydrogen, halogen, or a (C₁-C₆)alkyl, (C₁-C₆)alkoxy or a (C₁-C₆)alkoxy (C₁-C₆)alkyl group, wherein any one of which groups may be unsubstituted or substituted with one or more halogens; and

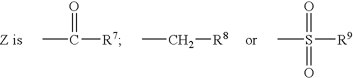

wherein R⁷ is hydrogen or (C₁-C₃)alkoxy; R⁸ is hydrogen, hydroxy, or (C₁-C₃)alkoxy; and R⁹ is (C₁-C₃)alkoxy.

In other preferred embodiments, X is oxygen; R¹ is hydrogen; R² is hydrogen or fluorine; R⁷ is methoxy; R⁸ is hydroxy; and R⁹ is methyl.

In another preferred embodiment the compound of Formula I is independently any one or more than one compound selected from the group consisting of:

(7R, 9aS)-trans-6-(2-Benzo[d]isoxazol-3-yl-octahydro-pyrido[1,2-a]pyrazin-7-ylmethoxy)-nicotinic acid methyl ester;

(7R, 9aS)-trans-6-(2-Benzo[d]isoxazol-3-yl-octahydro-pyrido[1,2-a]pyrazin-7-ylmethoxy)-pyridin-3-yl]-methanol;

(7R, 9aS)-trans-Methanesulfonic acid 6-(2-benzo[d]isoxazol-3-yl-octahydro-pyrido[1,2-a]pyrazin-7-ylmethyoxy)-pyridin-2-ylmethyl ester;

(7R, 9aS)-trans-Methanesulfonic acid 6-[2-(5-fluoro-benzo[d]isoxazol-3-yl)-octahydro-pyrido[1,2-a]pyrazin-7-ylmethoxy]-pyridin-2-ylmethyl ester;

(7R, 9aS)-trans-6-(2-Benzo[d]isoxazol-3-yl-octahydro-pyrido[1,2-a]pyrazin-7-ylmethoxy)-pyridine-2-carboxylic acid methyl ester;

(7R, 9aS)-trans-6-(2-Benzo[d]isoxazol-3-yl-octahydro-pyrido[1,2-a]pyrazin-7-ylmethoxy)-pyridin-2-yl]-methanol;

(7R, 9aS)-trans-{6-[2-(5-Fluoro-benzo[d]isoxazol-3-yl)-octahydro-pyrido[1,2-a]pyrazin-7-ylmethoxy]-pyridin-2-yl}-methanol;

(7R, 9aS)-trans-6-[2-(5-Fluoro-benzo[d]isoxazol-3-yl)-octahydro-pyrido[1,2-a]pyrazin-7-ylmethoxy]-pyridine-2-carboxylic acid ester;

(7R, 9aS)-cis-6-(2-Benzo[d]isoxazol-3-yl-octahydro-pyrido[1,2-a]pyrazin-7-ylmethoxy)-pyridine-2-carboxylic acid methyl ester;

(7R, 9aS)-cis-6-(2-Benzo[d]isoxazol-3-yl-octahydro-pyrido[1,2-a]pyrazin-7-ylmethoxy)-pyridin-2-yl]-methanol;

(7R, 9aS)-cis-Methanesulfonic acid 6-(2-benzo[d]isoxazol-3-yl-octahydro-pyrido[1,2-a]pyrazin-7-ylmethyoxy)-pyridin-2-ylmethyl ester;

(7R, 9aS)-trans-5-(2-Benzo[d]isoxazol-3-yl-octahydro-pyrido[1,2-a]pyrazin-7-ylmethoxy)-pyridine-2-carboxylic acid methyl ester;

(7R, 9aS)-trans-[5-(2-Benzo[d]isoxazol-3-yl-octahydro-pyrido[1,2-a]pyrazin-7-ylmethoxy)-pyridin-2-yl]-methanol; and

(7R, 9aS)-trans-Methanesulfonic acid 5-(2-benzo[d]isoxazol-3-yl-octahydro-pyrido[1,2-a]pyrazin-7-ylmethoxy)-pyridin-2-ylmethyl ester.

In yet another preferred embodiment, the compound of Formula I is aminomethylpyridinyloxymethyl/benzisoxazole.

In the compound of Formula I, in any ring formed by NR³R⁴: (a) there is not more than one ring oxygen atom; (b) no hydroxy, alkoxy, alkoxyalkyl, cyano, amino, or alkylamino moiety bonded directly to any ring nitrogen atom; and (c) no ring carbon that is double bonded to another ring carbon and no part of an aromatic ring system can be bonded to a ring oxygen atom or ring nitrogen atom.

Unless otherwise indicated, the following terms and related variations of same as used herein representatiyely have the meanings ascribed:

“Halogen” and “halo” and the like includes fluoro, chloro, bromo, and iodo.

“Alkyl” including as appears in the terms “alkoxy,” “alkyoxyalkyl,” and “aralkyl,” includes saturated monovalent hydrocarbon radicals having straight or branched moieties. Examples of alkyl groups include, but are not limited to, methyl, ethyl, n-propyl, isopropyl, and t-butyl.

“Methylene” refers to the divalent radical —(CH₂)ₚ- where p is 1 (methylene), 2 (dimethylene) or 3 (trimethylene).

“Cycloalkyl” includes non-aromatic saturated cyclic alkyl moieties wherein alkyl is as defined above. Examples of cycloalkyl include, but are not limited to, cyclopropyl, cyclobutyl, cyclopentyl, cyclohexyl, and cycloheptyl; and bicycloalkyl and tricycloalkyl groups that are non-aromatic saturated carbocyclic groups consisting of two or three rings respectively, wherein said rings share at least one carbon atom. For purposes of the present invention, and unless otherwise indicated, bicycloalkyl groups include spiro groups and fused ring groups. Examples of bicycloalkyl groups include, but are not limited to, bicyclo-[3.1.0]-hexyl, bicyclo-[2.2.1]-hept-1-yl, norbornyl, spiro[4.5]decyl, spiro[4.4]nonyl, spiro[4.3]octyl, and spiro[4.2]heptyl. An example of a tricycloalkyl group is adamantanyl. Cycloalkyl groups also include groups that are substituted with one or more oxo moieties. Examples of such groups with oxo moieties are oxocyclopentyl and oxocyclobutyl.

“Aryl” includes an organic radical derived from an aromatic hydrocarbon by removal of one hydrogen, such as phenyl, naphthyl, indenyl, indanyl, and fluorenyl; and fused ring groups wherein at least one ring is aromatic.

“Heterocyclic” refers to a cyclic group containing one or more heteroatoms, preferably from one to four heteroatoms, each selected from O, S and N. Heterocyclic groups also include ring systems substituted with one or more oxo moieties. Examples of heterocyclic groups are aziridinyl, azetidinyl, pyrrolidinyl, piperidinyl, azepinyl, piperazinyl, 1,2,3,6-tetrahydropyridinyl, oxiranyl, oxetanyl, tetrahydrofuranyl, tetrahydrothienyl, tetrahydropyranyl, tetrahydrothiopyranyl, morpholino, thiomorpholino, thioxanyl, pyrrolinyl, indolinyl, 2H-pyranyl, 4H-pyranyl, dioxanyl, 1,3-dioxolanyl, pyrazolinyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, quinolizinyl, quinuclidinyl, 1,4-dioxaspiro[4.5]decyl, 1,4-dioxaspiro[4.4]nonyl, 1,4-dioxa-spiro[4.3]octyl, and 1,4-dioxaspiro[4.2]heptyl.

“Heteroaryl” refers to aromatic groups containing one or more heteroatoms (O, S, or N), preferably from one to four heteroatoms. A multicyclic group containing one or more heteroatoms wherein at least one ring of the group is aromatic is a “heteroaryl” group. The heteroaryl groups of this invention can also include ring systems substituted with one or more oxo moieties. Examples of heteroaryl groups are pyridinyl, pyridazinyl, imidazolyl, pyrimidinyl, pyrazolyl, triazolyl, pyrazinyl, quinolyi, isoquinolyl, 1,2,3,4-tetrahydroguinolyl, tetrazolyl, furyl, thienyl, isoxazolyl, thiazolyl, oxazolyl, isothiazolyl, pyrrolyl, indolyl, benzimidazolyl, benzofuranyl, cinnolinyl, indazolyl, indolizinyl, phthalazinyl, triazinyl, 1,2,4-trizainyl, 1,3,5-triazinyl, isoindolyl, 1-oxoisoindolyl, purinyl, oxadiazolyl, thiadiazolyl, furazanyl, benzofurazanyl, benzothiophenyl, benzotriazolyl, benzothiazolyl, benzoxazolyl, quinazolinyl, quinoxalinyl, naphthyridinyl, dihydroquinolyl, tetrahydroquinolyl, dihydroisoquinolyl, tetrahydroisoquinolyl, benzofuryl, furopyridinyl, pyrolopyrimidinyl, and azaindolyl.

The foregoing groups, as derived from the compounds listed above, may be bonded via a C atom or N atom where such is possible. For instance, a group derived from pyrrole may be pyrrol-1-yl (bonded via N) or pyrrol-3-yl (bonded via C). The terms referring to the groups also encompass all possible tautomers.

“Amino” includes moieties of the formula —NR⁵R⁶ wherein R⁵ and R⁶ are each independently hydrogen or (C₁-C₄)alkyl.

“Treatment” and “treating” refers to reversing, alleviating, inhibiting the progress of, or preventing the disorder or condition to which such term applies, or one or more symptoms of such condition or disorder. As used herein, the term also encompasses, depending on the condition of the patient, preventing the disorder, including preventing onset of the disorder or of any symptoms associated therewith, as well as reducing the severity of the disorder or any of its symptoms prior to onset. “Treating” as used herein refers also to preventing a recurrence of a disorder. The term “treatment”, as used herein, refers to the act of treating, as “treating” is defined immediately above.

“Mammal” refers to any member of the class “Mammalia”, including, but not limited to, humans, dogs, and cats.

“Modulating serotonergic neurotransmission” refers to increasing or improving, or decreasing or retarding the neuronal process whereby serotonin is released by a pre-synaptic cell upon excitation and crosses the synapse to stimulate or inhibit the post-synaptic cell.

“Chemical dependency” means an abnormal craving or desire for, or an addiction to a drug. Such drugs are generally taken by the affected individual by any of a variety of means, including oral, parenteral, nasal, or by inhalation. Examples of chemical dependencies treatable by the methods of the present invention are dependencies on alcohol, nicotine, cocaine, heroin, phenolbarbitol, and benzodiazepines (e.g. Valium®). “Treating a chemical dependency” as used herein, means reducing or alleviating such dependency and/or the craving therefor.

Without limitation, the disorders treated by the method of the invention are those wherein a ligand to D2, 5HT1B, and 5HT2A receptors, individually or in any combination, is indicated. In one aspect, the method comprises administering a therapeutically effective amount of a compound that is an inhibitor to at least two of the following receptors: D2, 5HT-1B, and 5HT2A. In another aspect of the invention, the method comprises administering a therapeutically effective amount of a D2/5HT1-B/5HT-2A inhibitor. In yet another aspect, the method comprises administering a therapeutically effective amount of a D2/5HT1B inhibitor having a ratio of D2:5HT1B inhibitory activity of about 20 or less, preferably about 10 or less; more preferably about 5 or less; and most preferably about 1.

Mood disorders include, without limitation, those wherein a ligand, e.g. an antagonist, an inverse agonist and/or a partial agonist and the like, to D2, 5HT1B, and 5HT2A receptors, either individually or any combinations thereof, are indicated. Thus in a preferred practice, the invention is a method that can treat disorders contemplated herein with a single compound, wherein inhibition of D2 and 5HT2A receptors is commonly indicated, and wherein inhibition of 5HT1B receptors is commonly indicated.

The diagnostic criteria for Somatization Disorder, as characterized in the DSM, is:
A. A history of many physical complaints beginning before age 30 years that occur over a period of several years and result in treatment being sought or significant impairment in social, occupational, or other important areas of functioning.
B. Each of the following criteria must have been met, with individual symptoms occurring at any time during the course of the disturbance:

(1) four pain symptoms: a history of pain related to at least four different sites or functions (e.g., head, abdomen, back, joints, extremities, chest, rectum, during menstruation, during sexual intercourse, or during urination)

(2) two gastrointestinal symptoms: a history of at least two gastrointestinal symptoms other than pain (e.g., nausea, bloating, vomiting other than during pregnancy, diarrhea, or intolerance of several foods)

(3) one sexual symptom: a history of at least one sexual or reproductive symptom other than pain (e.g., sexual indifference, erectile or ejaculatory dysfunction, irregular menses, excessive menstrual bleeding, vomiting throughout pregnancy)

(4) one pseudoneurological symptom: a history of at least one symptom or deficit suggesting a neurological condition not limited to pain (conversion symptoms such as impaired coordination or balance, paralysis or localized weakness, difficulty swallowing or lump in throat, aphonia, urinary retention, hallucinations, loss of touch or pain sensation, double vision, blindness, deafness, seizures; dissociative symptoms such as amnesia; or loss of consciousness other than fainting)
C. Either (1) or (2):

(1) after appropriate investigation, each of the symptoms in Criterion B cannot be fully explained by a known general medical condition or the direct effects of a substance (e.g., a drug of abuse a medication)

(2) when there is a related general medical condition, the physical complaints or resulting social or occupational impairment are in excess of what would be expected from the history, physical examination, or laboratory findings
D. The symptoms are not intentionally produced or feigned (as in Factitious Disorder or Malingering).

The diagnostic criteria for Borderline Personality Disorder, as characterized by the DSM, is a pervasive pattern of instability of interpersonal relationships, self image, and affects, and marked impulsivity that begins by early adulthood and is present in a variety of contexts, as indicated in five (or more) of the following:
(1) frantic efforts to avoid real or imagined abandonment
(2) a pattern of unstable and intense interpersonal relationships characterized by alternating between extremes of idealization and devaluation
(3) identity disturbance: markedly and persistently unstable self-image or sense of self
(4) impulsivity in at least two areas that are potentially self-damaging (e.g., spending, sex, substance abuse, reckless driving, binge eating)
(5) recurrent suicidal behavior, gestures, or threats, or self-mutilating behavior
(6) affective instability due to a marked reactivity of mood (e.g., intense episodic dysphoria, irritability, or anxiety usually lasting a few hours and only rarely more than a few days)
(7) chronic feelings of emptiness
(8) inappropriate, intense anger or difficulty controlling anger (e.g., frequent displays of temper, constant anger, recurrent physical fights)
(9) transient, stress-related paranoid ideation or severe dissociative symptoms

The diagnostic criteria for Narcissistic Personality Disorder as characterized in the DSM is a pervasive pattern of grandiosity (in fantasy or behavior), need for admiration, and lack of empathy, beginning by early adulthood and present in a variety of contexts, as indicated by five (or more) of the following:
(1) has grandiose sense of self-importance (e.g., exaggerates achievements and talents, expects to be recognized as superior without commensurate achievements)
(2) is preoccupied with fantasies of unlimited success, power, brilliance, beauty, or ideal love
(3) believes he or she is “special” and unique and can only be understood by, or should associate with, other special or high-status people (or institutions)
(4) requires excessive admiration
(5) has a sense of entitlement, i.e., unreasonable expectations of especially favorable treatment or automatic compliance with his or her expectations
(6) is interpersonally exploitive, i.e., takes advantage of others to achieve his or her own ends
(7) lacks empathy: is unwilling to recognize or identify with the feelings and needs of others
(8) is often envious of others or believes that others are envious of him or her
(9) shows arrogant, haughty behaviors or attitudes

The diagnostic criteria for Antisocial Personality Disorder as characterized in the DSM is:

A. A pervasive pattern of disregard for and violation of the rights of others occurring since age 15 years, as indicated by three (or more) of the following:
(1) failure to conform to social norms with respect to lawful behaviors as indicated by repeatedly performing acts that are grounds for arrest
(2) deceitfulness, as indicated by repeated lying, use of aliases, or conning others for personal profit or pleasure
(3) impulsivity or failure to plan ahead
(4) irritability and aggressiveness, as indicated by repeated physical fights or assaults
(5) reckless disregard for safety of self or others
(6) consistent irresponsibility, as indicated by repeated failure to sustain consistent work behavior or honor financial obligations
(7) lack of remorse, as indicated by being indifferent to rationalizing having hurt, mistreated, or stolen from another

B. The individual is at least age 18 years.

C. There is evidence of Conduct Disorder with onset before age 15 years.

D. The occurrence of antisocial behavior is not exclusively during the course of Schizophrenia or a Manic Episode.

Suicidal ideation, as defined by the National Center of Health Statistics, is having thoughts of suicide or of taking action to end one's own life. Suicidal ideation includes all thoughts of suicide, both when the thoughts include a plan to commit suicide and when they do not include a plan. Suicidal ideation is measured in the Youth Risk Behavior Survey by the question “During the past 12 months, did you ever seriously consider suicide?”

The compounds of Formula I can also be used in combination with other drugs, e.g. those conventionally used to treat CNS disorders. For example, the compounds of Formula I can be used in combination with ziprasidone and like compounds; or with a 5HT re-uptake inhibitor and like compounds.

Chemical dependencies include, for example, alcohol, amphetamine, cocaine, opiate, and nicotine addiction.

The present invention also relates to a method for treating a disorder or condition contemplated by the invention which is treatable by modulating serotonergic neurotransmission in a mammal, comprising administering to a mammal in need of such treatment a therapeutically effective amount of the compound having Formula I.

The present invention also relates to a method of treating a disorder or condition in a mammal contemplated by the invention comprising administering to a mammal in need of said treatment a therapeutically effective amount of a compound of Formula I and a pharmaceutically acceptable carrier.

The present invention also relates to a method of treating a mood disorder selected from the group consisting of Somatization Disorder, Borderline Personality Disorder, Narcissistic Personality Disorder, Suicidal Ideation, and Antisocial Personality Disorder, in a mammal comprising administering to said mammal a therapeutically effective amount of a compound which has at least 80% antagonism, or inverse agonist to each of D2, 5HT1B, and 5HT2A receptors, wherein said mammal is in need of said treatment.

The present invention also relates to a method of treating a mood disorder selected from the group consisting of Somatization Disorder, Borderline Personality Disorder, Narcissistic Personality Disorder, Suicidal Ideation, and Antisocial Personality Disorder in a mammal comprising administering to said mammal a therapeutically effective amount of a D2, 5HT1B inhibitor having effective inhibitory activity with an in vivo effective Ki of no more than 15 nM at each of said receptors, wherein said mammal is in need of such said treatment.

Examples of preferred compounds of the Formula I are those having the absolute stereochemical configuration defined as (7R, 9aS)-trans. Examples of preferred compounds of the Formula I are those having the absolute stereochemical configuration defined as (7S, 9aS)-cis.

U.S. Ser. No. 10/800,328 discloses methods of how to prepare the compounds of Formula I, which is incorporated herein by reference. It will be appreciated that other methodology or variations may be employed and are contemplated.

The compounds of Formula I which are basic in nature are capable of forming a wide variety of different salts with various inorganic and organic acids. Although such salts must be pharmaceutically acceptable for administration to animals, it is often desirable in practice to initially isolate a compound of the Formula I from the reaction mixture as a pharmaceutically unacceptable salt and then simply convert the latter back to the free base compound by treatment with an alkaline reagent, and subsequently convert the free base to a pharmaceutically acceptable acid addition salt. The acid addition salts of the base compounds of this invention are readily prepared by treating the base compound with a substantially equivalent amount of the chosen mineral or organic acid in an aqueous solvent medium or in a suitable organic solvent such as methanol or ethanol. Upon careful evaporation of the solvent, the desired solid salt is obtained.

The acids which are used to prepare the pharmaceutically acceptable acid addition salts of the base compounds of this invention are those which form non-toxic acid addition salts, e.g., salts containing pharmacologically acceptable anions, such as hydrochloride, hydrobromide, hydroiodide, nitrate, sulfate or bisulfate, phosphate or acid phosphate, acetate, lactate, citrate or acid citrate, tartrate or bitartrate, succinate, maleate, fumarate, gluconate, saccharate, benzoate, methanesulfonate and pamoate, i.e., 1,1′-methylene-bis-(2-hydroxy-3-naphthoate), salts.

The compounds and components of this invention should be considered independent and or capable of being combined in any fashion. Definitions in the specification and claims may be independent, dependent or multiply dependent according to their description.

The compounds of Formula I may advantageously be used in conjunction with one or more other therapeutic agents, for instance, different antidepressant agents such as tricyclic antidepressants (e.g., amitriptyline, dothiepin, doxepin, trimipramine, butripyline, clomipramine, desipramine, imipramine, iprindole, lofepramine, nortriptyline or protriptyline), monoamine oxidase inhibitors (e.g., isocarboxazid, phenelzine or tranylcyclopramine) or 5-HT re-uptake inhibitors (e.g., fluvoxamine, sertraline, fluoxetine or paroxetine), and/or with antiparkinsonian agents such as dopaminergic antiparkinsonian agents (e.g., levodopa, preferably in combination with a peripheral decarboxylase inhibitor e.g., benserazide or carbidopa, or with a dopamine agonist e.g., bromocriptine, lysuride or pergolide). It may also be used with acetocholinesterases such as donepezil. It is to be understood that the present invention covers the use of a compound of Formula I or a physiologically acceptable salt or solvate thereof in combination with one or more other therapeutic agents.

The compound of Formula I and the pharmaceutically acceptable salts thereof, in combination with a 5-HT re-uptake inhibitor (e.g. fluvoxamine, sertraline, fluoxetine or paroxetine), preferably sertraline, or a pharmaceutically acceptable salt or polymorph thereof is referred herein to as “the active combination”.

Serotonin (5-HT) re-uptake inhibitors, preferably sertraline, exhibit positive activity against depression; chemical dependencies; anxiety disorders including panic disorder, generalized anxiety disorder, agoraphobia, simple phobias, social phobia, and post-traumatic stress disorder; obsessive-compulsive disorder; avoidant personality disorder and premature ejaculation in mammals, including humans, due in part to their ability to block the synaptosomal uptake of serotonin.

Sertraline, (1S-cis)-4-(3,4-dichlorophenyl)-1,2,3,4-tetrahydro-N-methyl-1-naphthalenamine, has the chemical formula C₁₇H₁₇NC₁₂; its synthesis is described in U.S. Pat. No. 4,536,518 incorporated herein by reference. Sertraline hydrochloride is useful as an antidepressant and anorectic agent, and is also useful in the treatment of depression, chemical dependencies, anxiety obsessive-compulsive disorders, phobias, panic disorder, posttraumatic stress disorder, and premature ejaculation.

Activity of the active combination can be determined by methods (1)-(4) below, which are described in Koe, B. et al., Journal of Pharmacology and Experimental Therapeutics, 226 (3), 686-700 (1983). Specifically, activity can be determined by studying (1) their ability to affect the efforts of mice in escaping a swim-tank (Porsolt mouse “behavior despair” test), (2) their ability to potentiate 5-hydroxytryptophan-induced behavioral symptoms in mice in vivo, (3) their ability to antagonize the serotonin-depleting activity of p-chloroamphetamine hydrochloride in rat brain in vivo, and (4) their ability to block the uptake of serotonin, norepinephrine and dopamine by synaptosomal rat brain cells in vitro. The ability of the active combination to counteract reserpine hypothermia in mice in vivo can be determined according to the methods described in U.S. Pat. No. No. 4,029,731.

The compounds of Formula I can be administered either alone or in combination with pharmaceutically acceptable carriers, in either single or multiple doses. Suitable pharmaceutical carriers include inert solid diluents or fillers, sterile aqueous solutions and various organic solvents. The pharmaceutical compositions formed thereby can then be readily administered in a variety of dosage forms such as tablets, powders, lozenges, liquid preparations, syrups, injectable solutions, and the like. These pharmaceutical compositions can optionally contain additional ingredients such as flavorings, binders, excipients and the like. Thus, the compound of the invention may be formulated for oral, buccal, intranasal, parenteral (e.g. intravenous, intramuscular or subcutaneous), transdermal (e.g. patch), or rectal administration, or in a form suitable for administration by inhalation or insufflation.

For oral administration, the pharmaceutical compositions may take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid).

For buccal administration, the composition may take the form of tablets or lozenges formulated in conventional manner.

The compounds of Formula I of the invention may be formulated for parenteral administration by injection, including using conventional catheterization techniques or infusion. Formulations for injection may be presented in unit dosage form, e.g., in ampules or in multi-dose containers, with an added preservative. They may take such forms as suspensions, solutions or emulsions in oily or aqueous vehicles, and may contain formulating agents such as suspending, stabilizing and/or dispersing agents. Alternatively, the active ingredient may be in powder form for reconstitution with a suitable vehicle, e.g., sterile pyrogen-free water, before use.

In a further aspect, the method of the invention uses compositions of matter suitable for administration to a human patient as a solution (e.g., as an injectable or intranasally), comprising an inclusion complex of a salt of the compounds of the invention in a material such as cyclodextrin. Advantageously, in a preferred embodiment said inclusion complex provides an amount of compound of at least 2.5 mgA/ml when the amount of compound provided by said complex is measured at a cyclodextrin concentration of 40% w/v in water. The inclusion complex of compound in cyclodextrin can first be isolated by drying, usually by lyophilization. The isolated dry inclusion complex can be stored at room temperature for periods up to two years and longer, and reconstituted into a product solution as needed. When a product solution is required, it can be made by dissolving the isolated inclusion complex in water (or other aqueous medium) in an amount sufficient to generate a solution of the required strength for oral or parenteral administration to patients. If parenteral administration is the chosen route of administration, intramuscular injection is preferred. The compounds may be formulated for fast dispersing dosage forms (fddf), which are designed to release the active ingredient in the oral cavity. These have often been formulated using rapidly soluble gelatin-based matrices. These dosage forms are well known and can be used to deliver a wide range of drugs. Most fast dispersing dosage forms utilize gelatin as a carrier or structure-forming agent. Typically, gelatin is used to give sufficient strength to the dosage form to prevent breakage during removal from packaging, but once placed in the mouth, the gelatin allows immediate dissolution of the dosage form. Alternatively, various starches are used to the same effect. The compounds of the invention may also be used in fast-dissolving, fast-disintegrating dosage forms such as those described in Expert Opinion in Therapeutic Patents, 11 (6), 981-986, by Liang and Chen (2001).

The compound of the invention may also be formulated in rectal compositions such as suppositories or retention enemas, e.g., containing conventional suppository bases such as cocoa butter or other glycerides.

For intranasal administration or administration by inhalation, the compound of Formula I is conveniently delivered in the form of a solution or suspension from a pump spray container that is squeezed or pumped by the patient or as an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the active compound. Capsules and cartridges (made, e.g., from gelatin) for use in an inhaler or insufflator may be formulated containing a powder mix of a compound of the invention and a suitable powder base such as lactose or starch.

A proposed dose of the active compounds of the invention for oral, parenteral or buccal administration to the average adult human for the treatment of the conditions referred to above (e.g., depression) is about 0.1 to about 200 mg of the active ingredient per unit dose which could be administered, for example, 1 to 4 times per day.

Aerosol formulations for treatment of the conditions referred to above (e.g., migraine) in the average adult human are preferably arranged so that each metered dose or “puff” of aerosol contains about 20 mg to about 1000 mg of the compound of the invention. The overall daily dose with an aerosol will be within the range of about 100 mg to about 10 mg. Administration may be several times daily, e.g. 2, 3, 4, or 8 times, giving for example, 1, 2, or 3 doses each time.

In connection with the use of the compound of Formula I with a 5-HT re-uptake inhibitor, preferably sertraline, for the treatment of subjects possessing any of the above conditions, it is to be noted that these may be administered either alone or in combination with pharmaceutically acceptable carriers by either of the routes previously indicated, and that such administration can be carried out in both single and multiple dosages. More particularly, the active combination can be administered in a wide variety of different dosage forms, i.e. they may be combined with various pharmaceutically-acceptable inert carriers in the form of tablets, capsules, lozenges, troches, hard candies, powders, sprays, aqueous suspension, injectable solutions, elixirs, syrups, and the like. Such carriers include solid diluents or fillers, sterile aqueous media and various non-toxic organic solvents, etc. Moreover; such oral pharmaceutical formulations can be suitably sweetened and/or flavored by means of various agents of the type commonly employed for such purposes. In general, the compounds of Formula I are present in such dosage forms at concentration levels ranging from about 0.5% to about 90% by weight of the total composition, i.e., in amounts which are sufficient to provide the desired unit dosage and a 5-HT re-uptake inhibitor, preferably sertraline, is present in such dosage forms at concentration levels ranging from about 0.5% to about 90% by weight of the total composition, i.e. in amounts which are sufficient to provide the desired unit dosage.

A proposed daily dose of the compound of the invention in the combination formulation (a formulation containing the compound of the invention and a 5-HT re-uptake inhibitor) for oral, parenteral, rectal or buccal administration to the average adult human for the treatment of the conditions referred to above is from about 0.01 mg to about 2000 mg, preferably from about 0.1 mg to about 200 mg of the active ingredient of Formula I per unit dose which could be administered, for example, 1 to 4 times per day.

A proposed daily dose of a 5-HT re-uptake inhibitor, preferably sertraline, in the combination formulation for oral, parenteral or buccal administration to the average adult human for the treatment of the conditions referred to above is from about 0.1 mg to about 2000 mg, preferably from about 1 mg to about 200 mg of the 5-HT re-uptake inhibitor per unit dose which could be administered, for example, 1 to 4 times per day.

A preferred dose ratio of sertraline to an active compound of this invention in the combination formulation for oral, parenteral or buccal administration to the average adult human for the treatment of the conditions referred to above is from about 0.00005 to about 20000; preferably from about 0.25 to about 2000.

Aerosol combination formulations for treatment of the conditions referred to above in the average adult human are preferably arranged so that each metered dose or “puff” of aerosol contains from about 0.01 mg to about 100 mg of the active compound of this invention, preferably from about 1 mg to about 10 mg of such compound. Administration may be several times daily, e.g. 2, 3, 4 or 8 times, giving for example, 1, 2 or 3 doses each time.

Aerosol formulations for treatment of the conditions referred to above in the average adult human are preferably arranged so that each metered dose or “puff” of aerosol contains from about 0.01 mg to about 2000 mg of a 5-HT re-uptake inhibitor, preferably sertraline, preferably from about 1 mg to about 200 mg of sertraline. Administration may be several times daily, for example 2, 3, 4 or 8 times, giving for example, 1, 2 or 3 doses each time.

As previously indicated, a 5-HT re-uptake inhibitor, preferably sertraline, in combination with compounds of Formula I, are readily adapted to therapeutic use as antidepressant agents. In general, these antidepressant compositions containing a 5-HT re-uptake inhibitor, preferably sertraline, and a compound of Formula I are normally administered in dosages ranging from about 0.01 mg to about 100 mg per kg of body weight per day of a 5-HT re-uptake inhibitor, preferably sertraline, preferably from about 0.1 mg to about 10 mg per kg of body weight per day of sertraline; with from about 0.001 mg. to about 100 mg per kg of body weight per day of a compound of Formula I, preferably from about 0.01 mg to about 10 mg per kg of body weight per day of a compound of Formula I, although variations will necessarily occur depending upon the conditions of the subject being treated and the particular route of administration chosen.

What is claimed is:

1. A method of treating a mood disorder selected from the group consisting of Somatization Disorder, Borderline Personality Disorder, Narcissistic Personality Disorder, Suicidal Ideation, and Antisocial Personality Disorder in a mammal comprising administering to said mammal a therapeutically effective amount of a compound having Formula I: 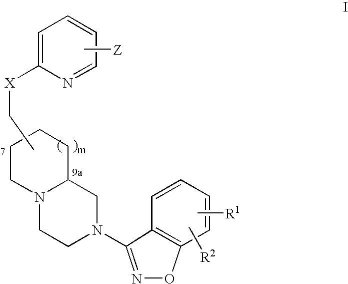
and pharmaceutically acceptable salts or solvates thereof, wherein
m is 0 or 1;
Z is 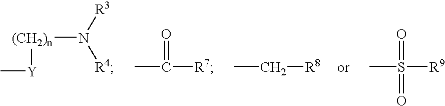
R⁷ is hydrogen or (C₁-C₃)alkoxy;
R⁸ is hydrogen, hydroxy, or (C₁-C₃)alkoxy;
R⁹ is (C₁-C₃)alkoxy;
X is oxygen or NR, wherein R is hydrogen or (C₁-C₆)alkyl;
Y is methylene when n is 0, 1 or 2;
or Y is oxygen, nitrogen or sulfur, when n is 2, 3 or 4;
R¹ and R² are each independently hydrogen, halogen, or a (C₁-C₆)alkyl, (C₁-C₆)alkoxy or a (C₁-C₆)alkoxy(C₁-C₆)alkyl group, wherein any one of which (C₁-C₆)alkyl, (C₁-C₆)alkoxy or (C₁-C₆)alkoxy(C₁-C₆)alkyl groups may be unsubstituted or substituted with one or more halogens;
R³ and R⁴ are each independently hydrogen, a (C₁-C₆)alkyl, a (C₃-C₇)cycloalkyl, or a 5 to 6 membered heterocyclic group, wherein any one of which (C₁-C₆)alkyl, (C₃-C₇)cycloalkyl, or 5 to 6 membered heterocyclic groups may be unsubstituted or substitituted with one or more substituents selected from the group consisting of (C₁-C₄)alkyl, (C₃-C₇)cycloalkyl, (C₁-C₄)alkoxy, (C₆-C₁₀)aryl, a 5 to 6 member heterocyclic, amino, halogen and hydroxy groups, wherein amino is NR⁵R⁶ where R⁵ and R⁶ are each independently hydrogen or (C₁-C₃)alkyl; or
R³ and R⁴, together with the nitrogen atom to which they are attached, form:
(i) a 3 to 7 membered saturated or unsaturated monocyclic ring; or
(ii) a 4 to 10 membered saturated or unsaturated polycyclic ring,
wherein said monocyclic or polycyclic ring optionally has one or two heteroatoms selected from nitrogen, oxygen and sulfur,
wherein any of said rings (i) or (ii) may be unsubstituted or substituted with one or more (C₁-C₄)alkyl, (C₁-C₄)alkoxy, (C₁-C₄)alkoxy(C₁-C₄)alkyl, (C₃-C₇)cycloalkyl, (C₆-C₁₀)aryl, (C₇ to C₁₃)aralkyl, a 5 to 10 membered heteroaryl, hydroxy, amino, cyano, or halogen groups.

2. The method of claim 1, wherein the compound of the invention has the formula: 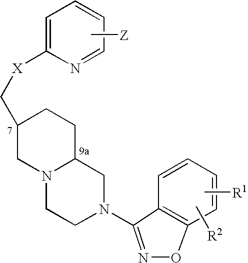
or a pharmaceutically acceptable salt or solvate thereof, wherein Z is 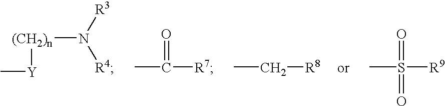
wherein R⁷ is hydrogen or (C₁-C₃)alkoxy;
R⁸ is hydrogen, hydroxy, or (C₁-C₃)alkoxy;
R⁹ is (C₁-C₃)alkoxy;
X is oxygen or NR, wherein R is hydrogen or (C₁-C₆)alkyl;
Y is methylene when n is 0, 1 or 2; or oxygen, nitrogen or sulfur when n is 2, 3 or 4;
R¹ and R² are each independently hydrogen, halogen, or a (C₁-C₆)alkyl, (C₁-C₆)alkoxy or a (C₁-C₆)alkoxy(C₁-C₆)alkyl group, wherein any one of which (C₁-C₆)alkyl, (C₁-C₆)alkoxy or (C₁-C₆)alkoxy(C₁-C₆)alkyl groups may be unsubstituted or substituted with one or more halogens;
R³ and R⁴ are each independently hydrogen, a (C₁-C₆)alkyl, a (C₃-C₇)cycloalkyl, or a 5 to 6 membered heterocyclic group, wherein any one of which (C₁-C₆)alkyl, (C₃-C₇)cycloalkyl, or 5 to 6 membered heterocyclic groups may be unsubstituted or substitituted with one or more of any of the following: (C₁-C₄)alkyl, (C₃-C₇)cycloalkyl, (C₁-C₄)alkoxy, (C₆-C₁₀)aryl, a 5 to 6 member heterocyclic, amino, halogen or hydroxy groups; or
R³ and R⁴ together with the nitrogen atom to which they are attached form:
(i) a 3 to 7 membered saturated or unsaturated monocyclic ring; or
(ii) a 4 to 10 membered saturated or unsaturated polycyclic ring,
wherein said monocyclic or polycyclic ring optionally has one or two additional heteroatoms selected from nitrogen, oxygen and sulfur,
wherein any of said rings (i) or (ii) may be unsubstituted or substituted with one or more substituents selected from (C₁-C₄)alkyl, (C₁-C₄)alkoxy, (C₁-C₄)alkoxy(C₁-C₄)alkyl, (C₃-C₇)cycloalkyl, (C₆-C₁₀)aryl, (C₇ to C₁₃)aralkyl, a 5 to 10 membered heteroaryl, hydroxy, amino, cyano, and halogen groups.

3. The method of claim 1 wherein the compound of Formula I has the following structure: 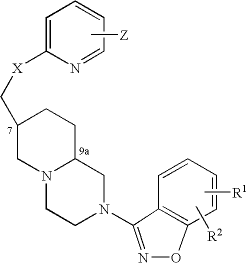
wherein Z is 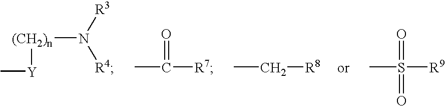
X is oxygen; n is 0; R¹ is hydrogen; R² is hydrogen or halogen; and R³ is hydrogen or a (C₁-C₃)alkyl.

4. The method of claim 3 wherein R² is hydrogen; R³ is hydrogen; and R⁴ is
a) a (C₁-C₆)alkyl group;
b) a (C₃-C₇)cycloalkyl group; or
c) a 6 to 6 member heterocyclic group,
wherein any one of which groups a), b) or c) may be unsubstituted or substitituted with one or more of any of the following: (C₁-C₄)alkyl, (C₃-C₇)cycloalkyl, (C₁-C₄)alkoxy, (C₆-C₁₀)aryl, a 5 to 6 member heterocyclic, amino, halogen or hydroxy groups.

5. The method of claim 3 wherein Z is 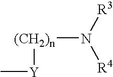
Y is methylene; and R⁴ is
a) a (C₁-C₄)alkyl which may be unsubstituted or substituted with one of the following: phenyl, cyclopropyl, methoxy, or substituted with a 5 to 6 membered heterocyclic, said heterocyclic having at least one nitrogen or oxygen atom;
b) an unsubstituted (C₃-C₇)cycloalkyl; or
c) a 5 to 6 membered heterocyclic group which can be unsubstituted or substituted with a (C₁-C₃)alkyl or a (C₁-C₃)alkoxy, said 5 to 6 member heterocyclic c) having at least one nitrogen atom and up to one other heteroatom selected from nitrogen, oxygen and sulfur.

6. The method of claim 5 wherein R⁴ is
a) an unsubstituted C₄ alkyl; a C₃ alkyl substituted with methoxy; a (C₁-C₂)alkyl substituted with phenyl or cyclopropyl; a (C₁-C₂)alkyl substituted with a 5 membered heterocyclic having a nitrogen or oxygen atom; or a (C₁-C₂)alkyl substituted with a 6 membered heterocyclic having at least one nitrogen;
b) an unsubstituted cyclopropyl; or
c) a 5 to 6 membered heterocyclic group which can be unsubstituted or substituted with a methyl or methoxy, said 5 to 6 membered ring c) having at least one nitrogen atom and up to one other heteroatom selected from nitrogen, oxygen and sulfur, said (C₁-C₃)alkyl is methyl and said (C₁-C₃)alkoxy is methoxy.

7. The method of claim 2 wherein R² is hydrogen; R³ is (C₁-C₃)alkyl; and R⁴ is
a) a (C₁-C₄)alkyl group; or
b) a (C₅-C₆)cycloalkyl group, wherein either of which groups a) or b) may be unsubstituted or substituted with one or more (C₁-C₃)alkoxy or amino groups.

8. The method of claim 1 wherein Z is 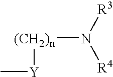
wherein Y is methylene; X is oxygen; n is 0; R¹ is hydrogen; R² is hydrogen; and R³ and R⁴ together with the nitrogen atom to which they are attached form i) a saturated non-aromatic 3 to 7 membered monocyclic ring, said ring i) being unsubstituted or substituted with one or more (C₁-C₄)alkyl, (C₁-C₄)alkoxy(C₁-C₄)alkyl, or hydroxy groups.

9. The method of claim 1 wherein Z is 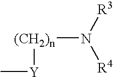
wherein Y is methylene; X is oxygen; n is 0; R¹ is hydrogen; R² is hydrogen; and R³ and R⁴ together with the nitrogen atom to which they are attached form an unsubstituted 5 to 6 membered heterocyclic ring, which heterocyclic ring, in addition to the nitrogen atom to which R³ and R⁴ are attached, has one additional nitrogen atom, or one sulfur atom, or one oxygen atom.

10. The method of claim 2 wherein Z is 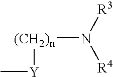
wherein Y is methylene; n is 0; R² is halogen; and R⁴ is
a) a (C₁-C₅ )alkyl;
b) a (C₃-C₆) cycloalkyl group, wherein any of which groups a) or b) can be unsubstituted or substituted with one or more of any of the following: cyclopropyl; halogen; hydroxy; a 5 to 6 membered heterocyclic group wherein said 5 to 6 membered heterocyclic group may be unsubstituted or substituted with one or more methyl groups; or phenyl wherein said phenyl may be unsubstituted or substituted with one or more halogens; or R⁴ is
c) a 5 membered heterocyclic group.

11. The method of claim 10 wherein R² is fluorine; and R³ is hydrogen or methyl.

12. The method of claim 2 wherein R² is halogen; and R³ and R⁴ together with the nitrogen atom to which they are attached form
i) a saturated 3 to 7 membered monocyclic ring, which monocyclic ring may be unsubstituted or substituted with one or more phenyl, (C₁-C₃)alkyl, or (C₁₋₄)alkoxy(C₁₋₄)alkyl groups; or
ii) a 5 to 6 membered monocyclic ring, which ring may be unsubstituted or substituted with one or more (C₁-C₃) alkyl groups, and which ring has one additional nitrogen or one oxygen atom.

13. The method of claim 1 wherein the compound of Formula I is selected from the group consisting of:
(7R, 9aS)-trans-2-Benzo[d]isoxazol-3-yl-7-(6-morpholin-4-ylmethyl-pyridin-2-yloxymethyl)-octahydro-pyrido[1,2-a]pyrazine;
(7R, 9aS)-trans-2-Benzo[d]isoxazol-3-yl-7-(5-piperidin-1-ylmethyl-pyridin-2-yloxymethyl)-octahydro-pyrido[1,2-a]pyrazine;
(7R, 9aS)-trans-2-(5-Fluoro-benzo[d]isoxazol-3-yl)-7-(5-pyrrolidin-1-ylmethyl-pyridin-2-yloxymethyl)-octahydro-pyrido[1,2-a]pyrazine;
(7R, 9aS)-trans-[6-(2-Benzo[d]isoxazol-3-yl-octahydro-pyrido[1,2-a]pyrazin-7-ylmethoxy)-pyridin-3-ylmethyl]-diethyl-amine;
(7R, 9aS)-trans-[6-(2-Benzo[d]isoxazol-3-yl-octahydro-pyrido[1,2-a]pyrazin-7-ylmethoxy)-pyridin-3-ylmethyl]-dimethyl-amine;
(7R, 9aS)-trans-[6-(2-Benzo[d]isoxazol-3-yl-octahydro-pyrido[1,2-a]pyrazin-7-ylmethoxy)-pyridin-3-ylmethyl]-ethyl-methyl-amine;
(7R, 9aS)-trans-[6-(2-Benzo[d]isoxazol-3-yl-octahydro-pyrido[1,2-a]pyrazin-7-ylmethoxy)-pyridin-3-ylmethyl]-(2-methoxy-1-methyl-ethyl)-amine;
(7R, 9aS)-trans-[6-(2-Benzo[d]isoxazol-3-yl-octahydro-pyrido[1,2-a]pyrazin-7-ylmethoxy)-pyridin-3-ylmethyl]-(2-methoxy-ethyl)-methyl-amine;
(7R, 9aS)-trans-[6-(2-Benzo[d]isoxazol-3-yl-octahydro-pyrido[1,2-a]pyrazin-7-ylmethoxy)-pyridin-3-ylmethyl]-cyclopentyl-methyl-amine;
(7R, 9aS)-trans-7-(5-Azetidin-1-ylmethyl-pyridin-2-yloxymethyl)-2-benzo[d]isoxazol-3-yl-octahydro-pyrido[1,2-a]pyrazine;
(7R, 9aS)-trans-2-Benzo[d]isoxazol-3-yl-7-[5-(2-methyl-aziridin-1-ylmethyl)-pyridin-2-yloxymethyl]-octahydro-pyrido[1,2-a]pyrazine;
(7R, 9aS)-trans-2-Benzo[d]isoxazol-3-yl-7-[5-(2-methoxymethyl-pyrrolidin-1-ylmethyl)-pyridin-2-yloxymethyl]-octahydro-pyrido[1,2-a]pyrazine;
(7R, 9aS)-trans-[6-(2-Benzo[d]isoxazol-3-yl-octahydro-pyrido[1,2-a]pyrazin-7-ylmethoxy)-pyridin-3-ylmethyl]-tert-butyl-amine;
(7S, 9aS)-cis-[6-(2-Benzo[d]isoxazol-3-yl-octahydro-pyrido[1,2-a]pyrazin-7-ylmethoxy)-pyridin-3-ylmethyl]-ethyl-methyl-amine;
(7S, 9aS)-cis-7-(5-Azetidin-1-ylmethyl-pyridin-2-yloxymethyl)-2-benzo[d]isoxazol-3-yl-octahydro-pyrido[1,2-a]pyrazine;
(7R, 9aS)-trans-[6-(2-Benzo[d]isoxazol-3-yl-octahydro-pyrido[1,2-a]pyrazin-7-ylmethoxy)-pyridin-2-ylmethyl]-dimethyl-amine;
(7R, 9aS)-trans-Cyclohexyl-{6-[2-(5-fluoro-benzo[d]isoxazol-3-yl)-octahydro-pyrido[1,2-a]pyrazin-7-ylmethoxy]-pyridin-2-ylmethyl}-amine;
(7R, 9aS)-trans-2-(Ethyl-{6-[2-(5-fluoro-benzo[d]isoxazol-3-yl)-octahydro-pyrido[1,2-a]pyrazin-7-ylmethoxy]-pyridin-2-ylmethyl}-amino)-ethanol;
(7R, 9aS)-trans-7-[6-(2,6-Dimethyl-piperidin-1-ylmethyl)-pyridin-2-yloxymethyl]-2-(5-fluoro-benzo[d]isoxazol-3-yl )-octahydro-pyrido[1,2-a]pyrazine;
(7R, 9aS)-trans-(1,2-Dimethyl-propyl)-{6-[2-(5-fluoro-benzo[d]isoxazol-3-yl)-octahydro-pyrido[1,2-a]pyrazin-7-ylmethoxy]-pyridin-2-ylmethyl}-amine;
(7R, 9aS)-trans-[6-(2-Benzo[d]isoxazol-3-yl-octahydro-pyrido[1,2-a]pyrazin-7-ylmethoxy)-pyridin-2-ylmethyl]-(2-methoxy-ethyl)-methyl-amine;
(7R, 9aS)-trans-1-[6-(2-Benzo[d]isoxazol-3-yl-octahydro-pyrido[1,2-a]pyrazin-7-ylmethoxy)-pyridin-3-ylmethyl]-(S)-pyrrolidin-3-ol;
(7R, 9aS)-trans-1-[6-(2-Benzo[d]isoxazol-3-yl-octahydro-pyrido[1,2-a]pyrazin-7-ylmethoxy)-pyridin-3-ylmethyl]-(R)-pyrrolidin-3-ol;
(7R, 9aS)-trans-2-Benzo[d]isoxazol-3-yl-7-[5-(2-methyl-pyrrolidin-1-ylmethyl)-pyridin-2-yloxymethyl]-octahydro-pyrido[1,2-a]pyrazine;
(7R, 9aS)-trans-1-[6-(2-Benzo[d]isoxazol-3-yl-octahydro-pyrido[1,2-a]pyrazin-7-ylmethoxy)-pyridin-3-ylmethyl]-piperidin-4-ol;
(7R, 9aS)-trans-Cyclopropyl-{6-[2-(5-fluoro-benzo[d]isoxazol-3-yl)-octahydro-pyrido[1,2-a]pyrazin-7-ylmethoxy]-pyridin-2-ylmethyl}-amine;
(7R, 9aS)-trans-Cyclopropylmethyl-{6-[2-(5-fluoro-benzo[d]isoxazol-3-yl)-octahydro-pyrido[1,2-a]pyrazin-7-ylmethoxy]-pyridin-2-ylmethyl}-amine;
(7R, 9aS)-trans-2-(5-Fluoro-benzo[d]isoxazol-3-yl)-7-[6-(4-methyl-piperazin-1-ylmethyl)-pyridin-2-yloxymethyl]-octahydro-pyrido[1,2-a]pyrazine;
(7R, 9aS)-trans-2-(5-Fluoro-benzo[d]isoxazol-3-yl)-7-(6-piperidin-1-ylmethyl-pyridin-2-yloxymethyl)-octahydro-pyrido[1,2-a]pyrazine;
(7R, 9aS)-trans-{6-[2-(5-Fluoro-benzo[d]isoxazol-3-yl)-octahydro-pyrido[1,2-a]pyrazin-7-ylmethoxy]-pyridin-2-ylmethyl}-dimethyl-amine;
(7R, 9aS)-trans-{6-[2-(5-Fluoro-benzo[d]isoxazol-3-yl)-octahydro-pyrido[1,2-a]pyrazin-7-ylmethoxy]-pyridin-2-ylmethyl}-(tetrahydro-furan-2-ylmethyl)-amine;
(7R, 9aS)-trans-7-[6-(2,5-Dimethyl-pyrrolidin-1-ylmethyl)-pyridin-2-yloxymethyl]-2-(5-fluoro-benzo[d]isoxazol-3-yl)-octahydro-pyrido[1,2-a]pyrazine;
(7R, 9aS)-trans-{6-[2-(5-Fluoro-benzo[d]isoxazol-3-yl)-octahydro-pyrido[1,2-a]pyrazin-7-ylmethoxy]-pyridin-2-ylmethyl}-[3-(4-methyl-piperazin-1-yl)-propyl]-amine;
(7R, 9aS)-trans-{6-[2-(5-Fluoro-benzo[d]isoxazol-3-yl)-octahydro-pyrido[1,2-a]pyrazin-7-ylmethoxy]-pyridin-2-ylmethyl}-pyrrolidin-1-yl-amine;
(7R, 9aS)-trans-7-(6-Azepan-1-ylmethyl-pyridin-2-yloxymethyl)-2-(5-fluoro-benzo[d]isoxazol-3-yl)-octahydro-pyrido[1,2-a]pyrazine;
(7S, 9aS)-cis-[6-(2-Benzo[d]isoxazol-3-yl-octahydro-pyrido[1,2-a]pyrazin-7-ylmethoxy)-pyridin-3-ylmethyl]-cyclohexyl-methyl-amine;
(7R, 9aS)-trans-1-[6-(2-Benzo[d]isoxazol-3-yl-octahydro-pyrido[1,2-a]pyrazin-7-ylmethoxy)-pyridin-2-ylmethyl]-(S)-pyrrolidin-3-ol;
(7R, 9aS)-trans-1-[6-(2-Benzo[d]isoxazol-3-yl-octahydro-pyrido[1,2-a]pyrazin-7-ylmethoxy)-pyridin-2-ylmethyl]-(R)-pyrrolidin-3-ol;
(7R, 9aS)-trans-2-Benzo[d]isoxazol-3-yl-7-(6-pyrrolidin-1-ylmethyl-pyridin-2-yloxymethyl)-octahydro-pyrido[1,2-a]pyrazine;
(7R, 9aS)-trans-[6-(2-Benzo[d]isoxazol-3-yl-octahydro-pyrido[1,2-a]pyrazin-7-ylmethoxy)-pyridin-2-ylmethyl]-benzyl-amine;
(7R, 9aS)-trans-2-Benzo[d]isoxazol-3-yl-7-(5-pyrrolidin-1-ylmethyl-pyridin-2-yloxymethyl)-octahydro-pyrido[1,2-a]pyrazine; and
(7S, 9aS)-cis-2-Benzo[d]isoxazol-3-yl-7-(5-pyrrolidin-1-ylmethyl-pyridin-2-yloxymethyl)-octahydro-pyrido[1,2-a]pyrazine.

14. The method of any one of claims 1-13, wherein the compound of Formula I is administered to said mammal in combination with another mood drug.

15. The method of claim 14, wherein said mood drug is selected form Norepinephrine Reuptake Inhibitors, corticotropin-releasing hormone (CRH) antagonists and Selective Serotonin Reuptake Inhibitors.

16. A method of treating a mood disorder selected from the group consisting of Somatization Disorder, Borderline Personality Disorder, Narcissistic Personality Disorder, Suicidal Ideation, and Antisocial Personality Disorder in a mammal comprising administering to said mammal a therapeutically effective amount of a D2, and 5HT1B inhibitor having effective inhibitory activity with an in vivo effective Ki of no more than 15 nM at each of said receptors, wherein said mammal is in need of such said treatment.
